(19) United States
(12) Patent Application Publication
(10) Pub. No.: US 2010/0185152 A1
(43) Pub. Date: Jul. 22, 2010

(54) Dosing Operation In A Medical Device

(75) Inventors: Andre Larsen, Dragor (DK); Bo Vestergaard Jensen, Bronshoj (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 11/814,498

(22) PCT Filed: Feb. 3, 2006
(86) PCT No.: PCT/EP06/50643

§ 371 Date: Apr. 1, 2010

(51) Int. Cl. A61M 5/00

(52) U.S. Cl. 604/154

(57) ABSTRACT

This invention relates to a method of controlling a dosing operation where a piston in a medical device is moved to a desired position, the dosing operation applies a motor to provide a force from the piston to expel a dose of a liquid medicament, said method comprising the steps of: moving the piston, at a first fixed speed, to a first position of the movement, moving the piston, at a decreasing speed, from said first position to a second position of the movement, and moving the piston, at a second fixed speed, from said second position to the desired position of the movement. The method further comprises the step of letting a fixed waiting time pass, when the piston has reached the desired position. This enables for a precise movement and stop of the piston leading to a precise dose, minimized post dripping and to that only a fixed and short waiting time need to go before a user can withdraw an injection needle of the medical device from his skin.

The present invention relates to the control of a dosing operation in a medical device.

When a liquid medicament is to be supplied various way are possible for the person needing the medicament. For example the liquid medicament could be supplied in a vial, from which the patient could suck the appropriate dose subsequent to an injection.

It is well known in the art that liquid medicament can be supplied in prefilled cartridges. Such a cartridge is then to be inserted into a syringe, where after the appropriate dose is set on the syringe subsequent to an injection by means of a needle.

U.S. Pat. No. 6,340,357 discloses a drug delivery device wherein a dose to be apportioned from a cartridge is set by changing the relative position of co-operating dose setting elements and is injected by pressing a button until this button abuts a stop. By operation of count up or count down buttons the dose is set and read into an electronic circuit comprising a microprocessor and the dose setting movement of the dose setting elements relative to each other is performed by a motor controlled by the circuit in accordance with the read in dose. The set dose is shown on a display. The motor is further controlled to perform certain movements of the piston rod so as retraction of this rod when a cartridge is going to be changed an advancing of the piston rod to abutment with the piston after the cartridge has been changed and further to advance this piston to expel air from the cartridge.

U.S. Pat. No. 6,248,090 discloses a syringe having a dose setting mechanism, a button which can be operated to inject a set dose, a switch operated at a time between the start and completion of injection, and an electronic presentation of parameters such as the size of a set dose and the size of the last dose administered. The syringe also has a stop watch which is reset and started responsive to operation of the switch. The electronic presentation includes an indication of the number of hours elapsed from the activation of the switch, and may also include, for a predetermined period initially following the activation of the switch, a presentation of the number of seconds elapsed. The latter presentation can provide a visual indication to the patient of the length of time, after the injection button has been actuated to inject the dose, that the needle should remain inserted in the skin. Said length of time that the needle should remain inserted in the skin is from 4 to 10 seconds, preferably 6 seconds has been shown to be appropriate.

Typically, the patient will force a needle of the medical device into his skin, inject the dose and then wait a time before he withdraws the needle. This time needs to pass since post dripping has to take place and since the dose needs some time to be properly in place under the skin.

When a liquid medicament is to be supplied, it is important for the user that it supplied in the intended dose. E.g. if insulin—as the liquid medicament—is supplied in an amount less that the intended dose; it may lead to that the patient subsequently faces a too high blood sugar level.

Conversely, if insulin by accident or by an imprecise medical device is supplied in a too high amount as compared to the intended dose, this could have the effect that the patient subsequently faces a too low blood sugar level.

In both cases the too small amount or the too high amount of insulin will lead to—without the diabetic person is aware of it—that the intended dose of medication is not administered, which as a consequence means that a prescribed treatment with insulin is not followed.

Thus there is a need for a medical device with a secure and precise dosing mechanism.

Typically, in a medical device a movement of a piston is applied to expel (inject) the dose of the liquid medicament.

The above prior art devices involve the problem that when the piston is to travel a predetermined distance no means is provided to secure that the medication is dosed in a precise dose. Consequently, there is a need for a medical device with a secure and a precise movement of the piston as the dosing mechanism.

The need is fulfilled by a method of controlling a dosing operation where a piston in a medical device is moved to a desired position, in which during the dosing operation a motor is applied motor to provide a force from the piston to expel a dose of a liquid medicament, when said method comprises the steps of:

moving the piston, at a first fixed speed, to a first position of the movement,

moving the piston, at a decreasing speed, from said first position to a second position of the movement, and

moving the piston, at a second fixed speed, from said second position to the desired position of the movement, Sref.

It is an advantage of the invention that there will be precise movement and stop of the piston, especially since when the piston is about to reach its desired end position (the desired position of the movement, Sref), the piston is brought to a stop from the lowest possible speed, i.e. said second fixed speed. If—which is not the case—the motor driving the piston had to be brought to a stop from a relative high speed, all things considered, this would inevitably lead to a more varying position of the stop location, corresponding to an imprecise stop.

Furthermore, the movement and the stop of the piston are precise since the medical device does these operations in a controlled way (preferably by means of a microprocessor) as reflected in the three steps above. As consequence of a more precise stop position, the dose will be equally precise, since the dose is proportional to length of the movement of the piston.

As a consequence and as an advantage of the invention, less and a minimized post dripping is the case. This is the case since the piston after dosing is stopped subsequent to a relatively low dosing speed (second fixed speed). Therefore said piston is left in the stopped position in a relatively low compression (due to the relative low speed, the second fixed speed before stop). Subsequently the piston is to return to an uncompressed state from the compressed state, which leads to that certain of amount of liquid being expelled. This amount is minimized since—when the movement of the piston is stopped—the compressed state arose from the second fixed (which preferably is the minimum speed that the motor can run with) speed before stop.

Since a minimized post dripping is the case, this also contributes to the most precise dosing.

In an embodiment of the invention, said method further comprises the step of:

letting a fixed waiting time pass, when the piston has reached the desired position.

Hereafter said dosing operation is completed and the patient can withdraw the needle being sure that the liquid medicament is properly in place under the skin.

Typically the waiting time will be fixed and is selected as a fixed number from an interval between 3 to 6 seconds; preferably the fixed waiting time is set to 5 seconds.

Alternatively, the waiting time is set to about 2 seconds.

Alternatively, the waiting time is set to about 3 seconds.

Alternatively, the waiting time is set to about 4 seconds.

It is an advantage of the invention that the patient can withdraw the needle when the dose is expelled after only a short, but fixed waiting time. Furthermore, said waiting time is a fixed time since it is independent of the dose administered.

In a preferred embodiment of the invention, said liquid medicament is insulin, GLP-1 or human growth hormone, preferably insulin.

As discussed, the invention may be carried out on a medical device. In the present context, the term ‘medical device’ can mean an injector type device (such as a pen injector or a jet injector) for delivering a discrete dose of a liquid medication (possibly in the form of small drops), a medication pump for continuous delivery of a liquid medication.

U.S. Pat. No. 6,540,672, U.S. Pat. No. 6,656,114, US2002010432 and US2003032868 all disclose intelligent medical devices, which are hereby incorporated by reference in its entirety.

The invention will be explained more fully below in connection with preferred embodiments and with reference to the drawings, in which:

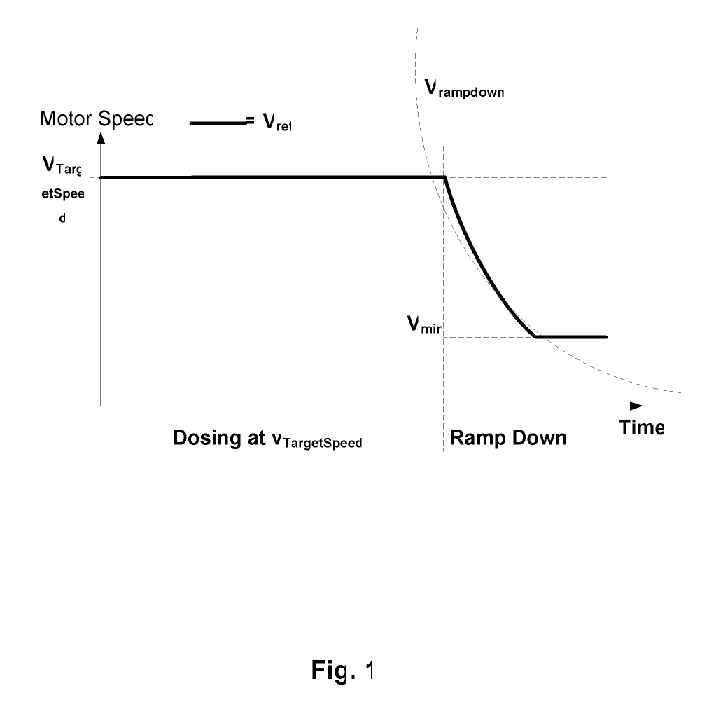

FIG. 1 shows an illustration of a ramp down method,

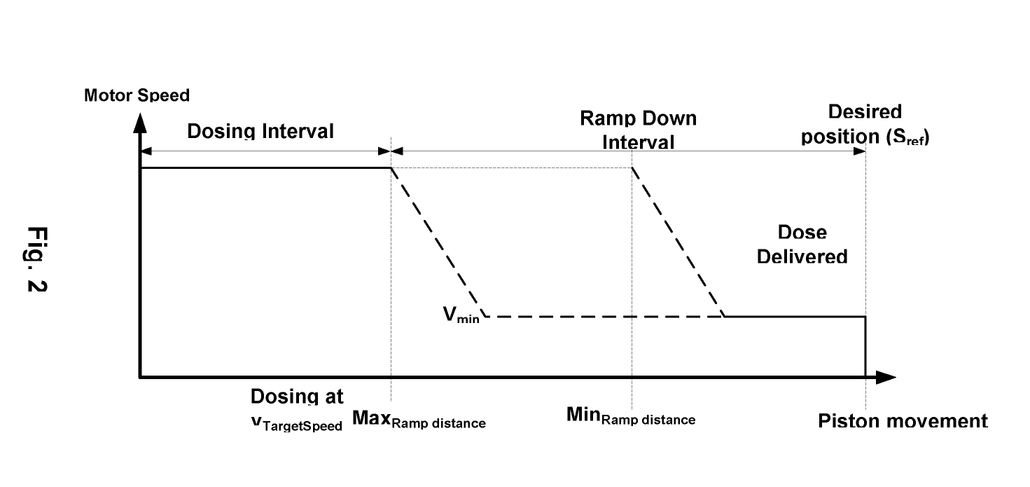

FIG. 2 shows ramping down motor speed before dose is dispensed in order to obtain fixed waiting time,

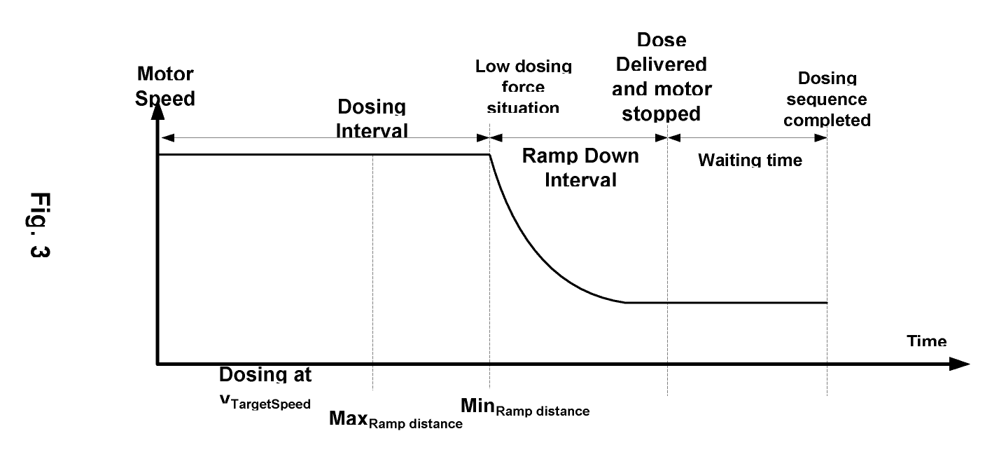

FIG. 3 shows motor speed versus time in a low dosing force situation,

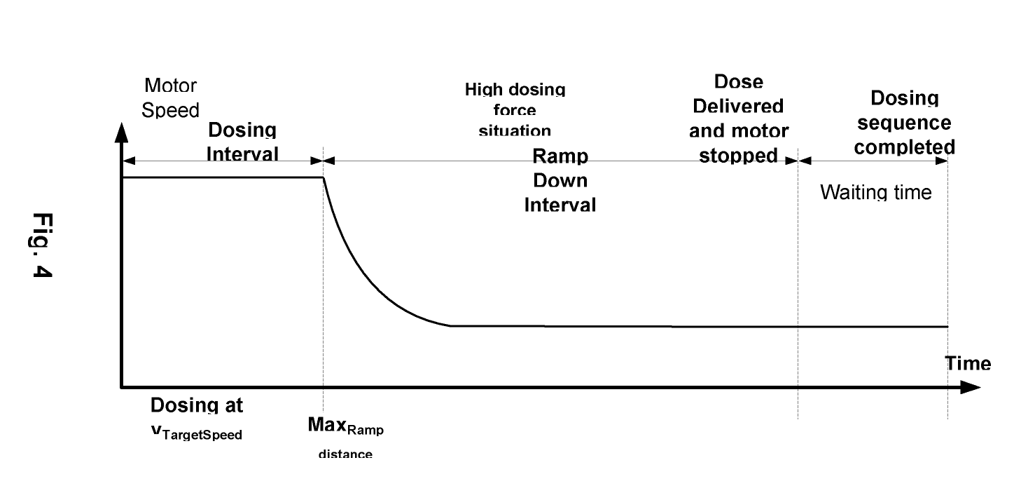

FIG. 4 shows motor speed versus time in a high dosing force situation,

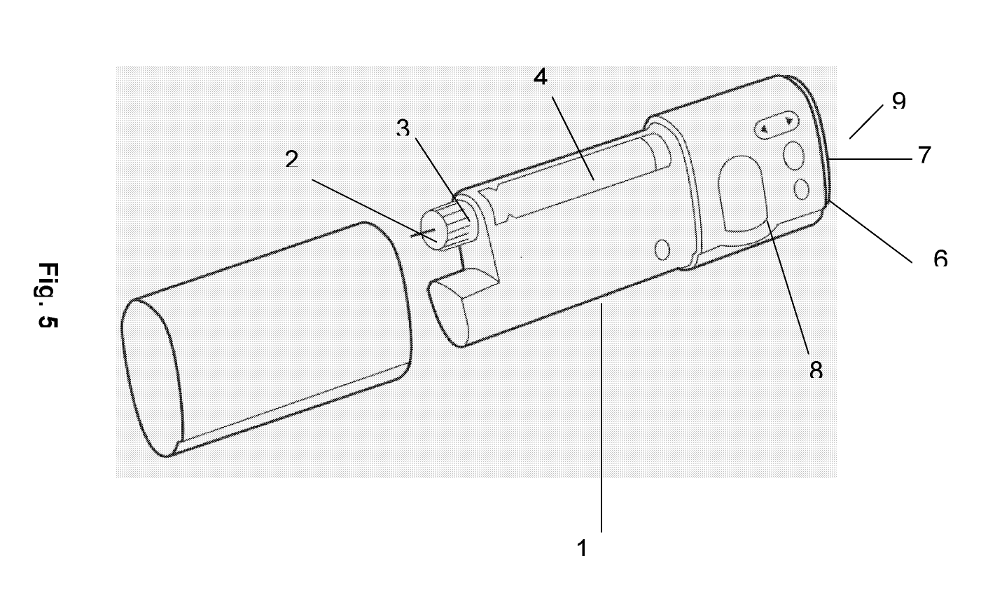

FIG. 5 shows an exemplary embodiment of a device,

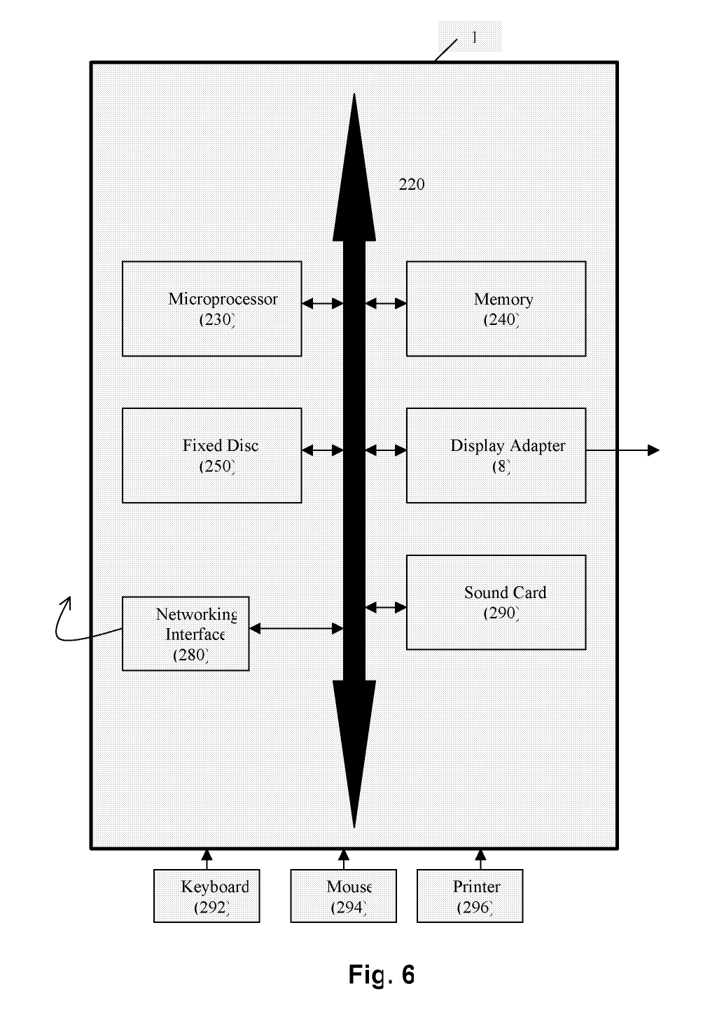

FIG. 6 shows an exemplary embodiment of the devices' electronic circuit, and

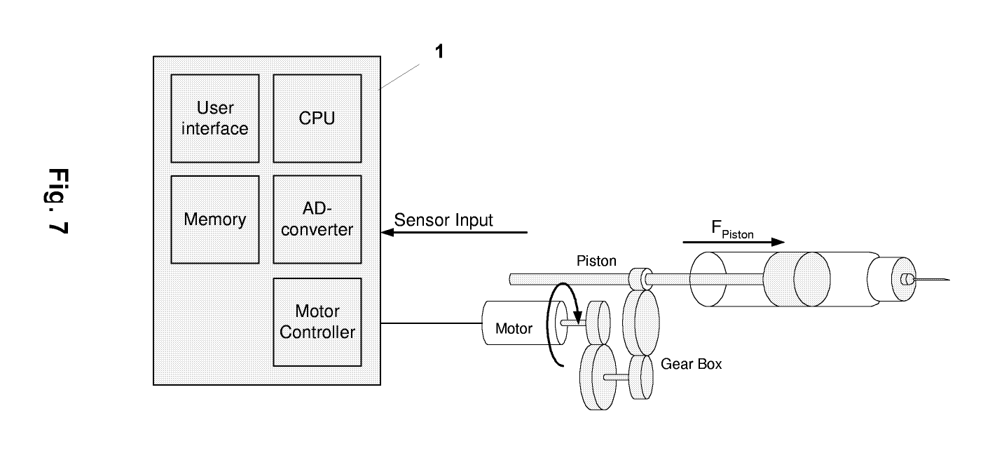

FIG. 7 shows another exemplary embodiment of the electronic circuit.

Throughout the drawings, the same reference numerals indicate similar or corresponding features, functions, etc.

FIG. 1 shows an illustration of a ramp down method. The bold line shows which speed reference, V_{ref} used at different times. As can be seen on the graph, the speed that is used is the smallest of ramp-down speed and nominal dosing speed (V_{TargetSpeed}), as long as the selected speed is larger than minimum speed.

It is appropriate in the control of the motor not to run the motor below V_{Min}, the minimum speed of said motor, thereby it is ensured that said motor does not run at a lower speed than said minimum speed. By a too low speed there is a risk that the piston is stuck due to a frictional force between the piston and a cartridge, e.g. a Penfill® cartridge.

When moving the piston, the microprocessor of the system controlling the piston movement is measuring the piston speed and compares it to a reference speed V_{ref.} V_{ref} is dependent on the remaining amount of movement in a selected movement. When ramping down, i.e. at a decreasing speed, V_{ref} is V_{rampdown} as will be discussed later.

The selection of V_{ref} during movement is important in order to obtain correct dose precision. V_{ref} is continuously updated, i.e. in the shown drawings, it follows the curve.

When moving the piston forward towards the desired position, where the selected dose is dispensed, the motor speed is ramped down in a controlled way. When ramping down the motor speed an algorithm in an exemplary embodiment of the invention is applied, the algorithm uses the remaining distance, i.e. the desired position minus the current position to determine the dosing speed.

In general, the term “ramping down” or “at a decreasing speed” could mean any linear, hyperbolic or any other speed decreasing function, e.g. when a curve is drawn of distance and speed, the curve expresses a falling speed versus the distance moved. The piston is controlled to start with a relatively high and fixed speed, it is then controlled to move at a decreasing speed, and finally the piston is then controlled such that it is forced to move at a relative low fixed speed; from the latter speed, which is the lowest speed during the movement of the piston, the piston is then controlled to a forced stop. Consequently, the speed just before the movement of the piston is stopped is lower than the speed when the dosing was commenced.

The compression of the cartridge, e.g. a Penfill® cartridge, piston during dosing is affecting the waiting time, which need to run, after dosing is completed. A large compression of the piston leads to more so called “post dripping”. If the piston during dosing is moved with a slow speed a lesser compression of the piston during its movement is the case, conversely running the piston during dosing with a higher speed a higher compression of the piston during its movement is the case.

If the piston during dosing is stopped subsequent to a relatively low dosing speed less post dripping will be the case since a relatively low compression (of the piston) is to return to an uncompressed state.

Conversely when the piston (residing in the cartridge, e.g. a Penfill® cartridge) after dosing with a relatively high dosing speed then is brought to a stop, this situation then leads to a high post dripping; this is the case since a relatively high compression of the piston is to return to an uncompressed state, which will make a volume of liquid—corresponding to the difference between the volume of the piston in the uncompressed state and the compressed state—drip out through the needle.

It is therefore desirable that the piston—after dosing—with a relatively low dosing speed in the completion of the movement is brought to a stop (from said relatively low dosing speed) since such situation leads to a relative low post dripping. This is the situation of the present invention.

Ramping down the motor, which ends in a relatively low dosing speed (e.g. V_{Min}, the minimum speed of said motor)—as discussed—reduces post dripping to a minimum, this is due to a smaller compression of the piston—and consequently a shorter waiting time only need to be the case before the needle can be withdrawn—after the selected dose has been dispensed—is therefore possible.

This means—in a practical application of the invention—that the patient can withdraw the needle when the dose is expelled (injected) after only a short, but fixed waiting time. Furthermore, said waiting time is fixed time since it is independent of the dose administered, and since it can be expected that the amount of liquid from the post dripping—which has to leave the medical device, e.g. through a needle—always will be in the same amount (of liquid) since the size of the compression of the piston also can be expected to be fixed. The latter is the case since the piston is to return to an uncompressed state from the compressed state arising from the relatively low dosing speed at the completion of the movement, i.e. just before the piston movement is brought to a stop having a speed of zero. The piston in the medical device is typically made of rubber and is compressible. If the piston is incompressible, the problem of post dripping does not arise.

In an embodiment of the invention, said fixed waiting time is selected as a fixed number from an interval between 2 to 6 seconds; preferably the fixed waiting time is set to 5 seconds.

In an embodiment of the invention, the fixed waiting time is set to about 2 seconds.

In an embodiment of the invention, the fixed waiting time is set to about 3 seconds.

In an embodiment of the invention, the fixed waiting time is set to about 4 seconds

In an embodiment of the invention, the fixed waiting time is set to about 5 seconds

Ramping down is done by decreasing the speed, starting from a constant nominal motor speed, e.g. V_{TargetSpeed}=1,488mm/s. This takes place during the completion of the movement. The invention may be applied when dosing or when the piston is moved in the opposite direction, i.e. when the piston is retracted as well.

The ramp down speed (V_{rampdown}) in an exemplary embodiment of the invention is calculated as a value dependent of the remaining distance (Sref−S) of the piston movement:

$${V}_{rampdown} = \frac{{S}_{ref} - S}{RampOnset} \cdot {V}_{TargetSpeed}$$

When the distance between the present position (S) and the desired end position (S_{ref}) is smaller than the specified Ramp Onset distance (calculated by Ramp Down onset algorithm), in this embodiment, the speed is proportional to the remaining distance, i.e. S_{ref}−S. If this speed is smaller than a specified minimum (Vₘᵢₙ), in this embodiment, then Vₘᵢₙ speed could be used, to make sure that the motor keeps running at a minimum speed.

A graph of the speed with respect to time in the normal case looks in principle like FIG. 1.

The reference speed, V_{ref} could be selected in the following way

Before dosing sequence starts:

Clear V_{ref}

During dosing the operation can be expressed in the following program pseudo code:

```
While S < Sref do
  Calculate V_{rampdown} as discussed above
  if (V_{TargetSpeed} > V_{rampdown}) (* start of ramping down *)
    if (V_{rampdown} > V_{Min})
      then V_{ref} = V_{rampdown}
    else
      then V_{ref} = V_{Min}  (* to ensure minimum speed of motor *)
  else
    V_{ref} = V_{TargetSpeed}  (* normal dosing before ramping down *)
```

Ramping down, i.e. moving the piston at a decreasing speed, is mainly used to obtain the required dosing precision, while the variation in Ramp Down onset based on the dosing force is used reduce post dripping. The reduced post dripping also contribute to improve the dosing precision, since post dripping is an undesired contribution to the dose.

FIG. 2 shows Ramping down motor speed before dose is dispensed in order to obtain fixed waiting time. The ramp down onset is based on measured dosing force, e.g. the motor current during dosing. A high motor current during dosing indicates high dosing force, which again means a larger compression of the piston compared to dosing with a low motor current. Large compression leads to a longer post dripping, which leads to a longer waiting time in order to ensure that the full selected dose is dispensed into the tissue.

The dosing force, F_{dose} is used in the following way to calculate the ramp down onset.

F_{dose}_{_{—}}_{Avg}=Average F_{dose} measured during dosing interval at fixed motor speed.

RampOnset=Position where ramping down is started

Min_{Ramp distance}=Minimum ramp onset. Constant e.g.=0.1488 mm.

Max_{Ramp distance}=Maximum ramp onset. Constant e.g.=0.8928 mm.

The Min_{Ramp distance} is typically set to 1 IU (Insulin Unit), whereas the Max_{Ramp distance} typically is set to 6 IU.

The typical ramp onset could be 2 IU corresponding to 2 times 0.1488 mm.

All distances (positions) are proportional to the dose size expressed in IU, Insulin Units.

F_{Min}=Minimum F_{dose}_{_{—}}_{avg}. Constant e.g.=10 N.

FIG. 3 shows motor speed versus. time in a low dosing force situation. At Max_{Ramp distance} Ramp down onset position is calculated. In the low dosing force case F_{dose}_{_{—}}_{avg} is close to F_{Min} and therefore the ramp onset position is set to Min_{Ramp distance}.

When the piston reaches MaxRamp distance from the desired position, Fdose_avg is found, the dosing force is currently measured during dosing interval at fixed motor speed, and on basis on a number of samples of the dosing force, the average value, i.e. Fdose_avg, of these samples is found. At this point in the dosing sequence the Ramp down onset is calculated as the following program pseudo code:

```
If Fdose_avg < FMin then
    Fdose_avg = FMin
RampOnset = Fdose_avg/Fmin * MinRamp distance
If RampOnset > MaxRamp distance then
    RampOnset = MaxRamp distance
```

FIG. 4 shows motor speed versus time in a high dosing force situation. At Max_{Ramp distance} Ramp down onset position is calculated. In the high dosing force case the F_{dose}_{_{—}}_{avg} is close to F_{Max} and therefore the ramp onset position is set to Max_{Ramp distance} and the total dosing time is longer compared to the low dosing force situation.

A high dosing force situation can be the case if the needle is rather thin, is partly blocked e.g. by a crystal or if the medicament has a high viscosity. Furthermore, the shape or wear of the piston could cause a high dosing force situation.

Stepwise description
User selects dose size, typical in IU, Insulin Units, the desired position S_{ref} is computed to be proportional to the dose size.
User inserts the needle in his skin
User activates dosing key
Motor controller or CPU calculates distance to move the piston. Desired position S_{ref}
Dosing start
At position Max_{Ramp distance} the ramp onset position is calculated using the ramp down onset formula
At position RampOnset, motor speed is ramped down using ramp down formula.
At the desired position Sref, the motor is stopped and waiting time starts
User waits until waiting symbol disappears and removes the needle from his skin

FIG. 5 discloses an exemplary embodiment of a device 1, e.g. a medical device having housing. An injection needle 2 is connected to a needle assembly 3 connected to the distal end of the housing and communicates with a container or reservoir 4, e.g. a cartridge or ampoule containing the medicine to be administered, e.g. an injection of basal or bolus insulin.

As an integral part of the device, a piston is provided at the end of a piston rod, which—in an embodiment of the invention—can be moved forth and back within the cylindrical shaped container 4, e.g. a Penfill® cartridge. The force for movement could be provided by a motor, e.g. a DC motor, a stepper motor, or an AC motor as well. When the piston is moved in the direction towards the injection needle the medicine to be administered can be expelled through said injection needle.

A plurality of operating buttons 5, 6, 7, 9 in an exemplary embodiment of the medical device is provided, these comprise a dose setting button 5 for setting a dose to be injected, an accept button 6 for accepting the dialled dose, an escape button 7 for moving backwards in the menu and an injection button 9.

In order to perform an injection the user could dial the size of the dose to be injected using the dial up/dial down button 5. As the dose is dialled, the size of the dose is displayed in the display 8. When the set dose is dialled to an adequate size, the user operates the accept button 7 thereby confirming the set dose. After having inserted the injection needle 2 into a tissue of a diabetic patient, the user operates the injection button 9 to release the set dose.

The release of the dose is performed as was discussed in FIGS. 1 to 4.

FIG. 6 discloses an exemplary embodiment of the devices' electronic circuit. Said device can be a medical device. This display of data can be implemented in a method which can be run on any general purpose device/computer system as shown in the figure, which shows its internal structure. The computer system (210), e.g. a device consists of various subsystems interconnected with the help of a system bus (220). The microprocessor (230) or CPU communicates and controls the functioning of other subsystems. Memory (240) helps the microprocessor in its functioning by storing instructions and data, e.g. such as medication of bolus insulin, by knowledge of the amount of insulin to be injected, the desired position Sref, i.e. the position the piston has to reach can be computed. All positions are proportional to the dose size expressed in IU, Insulin Units.

Since the piston—in an embodiment of the invention—can be moved in a cylindrical shaped container there is a linear relation ship between amount of insulin (# of IU) to be delivered and the length of the movement for the piston. Said amount (dose) of insulin to be delivered could be set by means of the dose setting button 5 as discussed in FIG. 5.

Fixed Drive (250) may be used to hold these data, e.g. in a database structure and instructions permanent in nature like the operating system and other programs, furthermore the fixed drive may contain data for a subsequent display. Display adapter (260) is used as an interface between the system bus and the display device (8), which is generally a monitor or a display. In other words, the display is interfaced with said processor, where the processor can be configured to cause the display to display various data as graphics, numbers text and any combinations thereof. This monitor or display can be used to display various data, such as medication of bolus insulin performed, to be performed from a treatment regimen at various point of time. Furthermore, sums of said data and other manipulations of said data can be shown of the display, as numbers, text, graphics, e.g. bar graph, pie chart, etc, the user of the device may determine what to show and how. The network interface (280) may be used to connect the computer with other computers on a network through wired or wireless means. These devices on the network can also be medical devices. These medical devices can be capable of storing patient related data such as drug dosage, point of times for drug dosage, e.g. for bolus insulin. These devices communicate with the computing device using various communication mediums. The communication means can be wired or wireless such as cable, RS232, Bluetooth, infrared etc using various communication protocols such as TCP/IP, SSL etc. The computer system might also contain a sound card (290). The system may be connected to various input devices like keyboard (292) and mouse (294) and output devices like printer (296). Various configurations of these subsystems are possible. It should also be noted that a device or system implementing the present invention might use less or more number of the subsystems than described above.

The number of devices can be expanded and customized as per the need to establish an efficient patient-doctor-relative-peer network. For example the computing system may periodically logon to a Local Area Network, or Internet to transmit the user readings, e.g. what doses of bolus insulin was administered at which point of times on a remote database server that might be used to generate reports or receive a treatment regimen for the diabetic patient from a different computing system such as that of a doctor, relative of the patient and the like. These computing devices can be general-purpose desktops or other variations such as laptop, cell phones, Personal Digital Assistants (PDAs), blood glucose meters, etc.

The method is incorporated in the aforementioned computing devices as by instructions in the software that are carried out by the computer system. Again, the software may be implemented as one or more modules for implementing the method.

In particular, the software may be stored in a computer readable medium, including the storage device or that is downloaded from a remote location via the interface and communications channel from the Internet or another network location or site. The computer system includes the computer readable medium having such software or program code recorded such that instructions of the software or the program code can be carried out. The use of the computer system preferably affects advantageous apparatuses for constructing a runtime symbol table for a computer program in accordance with the embodiments of the invention.

Said wireless transfer of data may be performed by means of transmission means, a network, e.g. a local area network (LAN), a wide area network (WAN), or any combination thereof, e.g. the Internet, an intranet, an extranet, or an on-line service.

Alternatively, the wireless transfer of data may be performed by means of IrDa, a Bluetooth communications standard or any other way as known in the art to transfer data wirelessly between two devices, e.g. a wireless client adapter, a wireless LAN adapter, etc. The wireless transfer may be implemented following a medical communication standard such as MICS, Medical Implant Communications Services or WMTS, i.e. the Wireless Medical Telemetry Service. Further, the transfer of data may be performed by means of a wireless LAN such as WI-FI using various standards such as 802.11a, 802.11 b or 802.11g or future developments thereof, e.g. Wimax, UWB (Ultra Wide Band) or ZigBee as a dynamic network implementation.

A computer readable storage medium may be a magnetic tape, an optical disc, a digital video disk (DVD), a compact disc (CD or CD-ROM), a mini-disc, a hard disk, a floppy disk, a smart card, a PCMCIA card, a ram stick, etc. or any other kind of media that provides a computer system with information regarding how instructions/commands should be executed.

FIG. 7 shows another exemplary embodiment of the electronic circuit. The user interface could correspond to the display 8 and the buttons shown 6, 7 including the arrow keys on FIG. 5. The memory could be used to hold counted signals, amount of insulin to be injected, digital force signals, etc. The AD converter could be used to convert an analogue current or voltage representing an analogous measured force into a digital force signal. The motor controller controls the speed, start/ stop and the direction of the motor. E.g. for a DC motor, a H-bridge as know in the art could be applied to start/stop and to control the direction of the motor, which consequently control the direction for the piston's movement. The gear-box could be used to convert (up/down) the motors' rotational speed (clockwise, counter clockwise) to a linear movement (forth or back) of the piston.

Any combination of the above-described elements in all possible variations thereof is encompassed by the invention unless otherwise indicated herein or otherwise clearly contradicted by context.

The use of the terms “a” and “an” and “the” and similar referents in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

All methods described herein can be performed in any suitable order unless otherwise indicated herein or otherwise clearly contradicted by context.

The description herein of any aspect or embodiment of the invention using terms such as “comprising”, “having,” “including,” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

This invention includes all modifications and equivalents of the subject matter recited in the aspects presented herein to the maximum extent permitted by applicable law.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

1. A method of controlling a dosing operation where a piston in a medical device is moved to a desired position of movement, the dosing operation applies a motor to provide a force from the piston to expel a dose of a liquid medicament, said method comprising:
moving the piston, at a first fixed speed, to a first position of the movement,
moving the piston, at a decreasing speed, from said first position to a second position of the movement, and moving the piston, at a second fixed speed, from said second position to the desired position S_{ref}.

2. The method according to claim 1, further comprising letting a fixed waiting time pass when the piston has reached the desired position.

3. The method according to claim 1, wherein said first position referred to as RampOnset and is calculated as Fdose_avg/Fmin*Min_{Ramp distance}, where Fdose_avg is the average dosing force during movement at said first fixed speed, Fmin is the minimum allowable average dosing force, and Min_{Ramp distance} is the minimum ramp onset position.

4. The method according to claim 1, wherein said second position is reached when said decreasing speed hits v_{Min}, the minimum speed of said motor, thereby ensuring that said motor does not run at a lower speed than said minimum speed.

5. The method according to claim 1, wherein said first fixed speed is a nominal dosing speed, V_{TargetSpeed}.

6. The method according to claim 3, wherein said decreasing speed V_{rampdown} is calculated as
$${V}_{rampdown} = \frac{{S}_{ref} - S}{RampOnset} \cdot {V}_{TargetSpeed} ,$$
S_{ref} is the desired position, V_{TargetSpeed} is the nominal dosing speed, and S is the current position of said movement from said first position to said second position.

7. The method according to claim 6, wherein said second fixed speed is V_{Min}, the minimum speed of said motor.

8. The method according to claim 1, wherein said liquid medicament is insulin, GLP-1 or human growth hormone.

9. The method according to claim 1, wherein said fixed waiting time is selected as a fixed number from an interval between 2 to 6 seconds.

10. The method according to claim 9, wherein the fixed waiting time is set to about 2 seconds.

11. The method according to claim 9, wherein the fixed waiting time is set to about 3 seconds.

12. The method according to claim 9, wherein the fixed waiting time is set to about 4 seconds.

13. The method according to claim 9, wherein the fixed waiting time is set to about 5 seconds.

14. The method according to claim 2, wherein said liquid medicament is insulin, GLP-1 or human growth hormone.

15. The method according to claim 3, wherein said liquid medicament is insulin, GLP-1 or human growth hormone.

16. The method according to claim 4, wherein said liquid medicament is insulin, GLP-1 or human growth hormone.

17. The method according to claim 5, wherein said liquid medicament is insulin, GLP-1 or human growth hormone.

18. The method according to claim 6, wherein said liquid medicament is insulin, GLP-1 or human growth hormone.

19. The method according to claim 7, wherein said liquid medicament is insulin, GLP-1 or human growth hormone.

20. The method according to claim 9, wherein said liquid medicament is insulin, GLP-1 or human growth hormone.
